(19) United States
(12) Patent Application Publication
(10) Pub. No.: US 2026/0240466 A1
(43) Pub. Date: Aug. 20, 2026

(54) BIOSENSOR BASED ON A NEAR FIELD COMMUNICATION USING DIRECT ELECTRON TRANSFER

(71) Applicants: THE UNIVERSITY OF NORTH CAROLINA AT CHAPEL HILL, Chapel Hill, NC (US); ARKRAY, INC., Kyoto-shi, Kyoto (JP)

(72) Inventors: Koji SODE, Chapel Hill, NC (US); David PROBST, Chapel Hill, NC (US)

(73) Assignees: THE UNIVERSITY OF NORTH CAROLINA AT CHAPEL HILL, Chapel Hill, NC (US); ARKRAY, INC., Kyoto-shi, Kyoto (JP)

(21) Appl. No.: 19/489,519

(22) PCT Filed: May 31, 2024
(86) PCT No.: PCT/US2024/032023

§ 371 Date: Dec. 2, 2025

Related U.S. Application Data
(60) Provisional application No. 63/505,828, filed on Jun. 2, 2023.

(51) Int. Cl. A61B 5/1486; A61B 5/145

(52) U.S. Cl. CPC A61B 5/14865; A61B 5/14532; A61B 5/14546

(57) ABSTRACT

The present disclosure relates to a biosensor comprising: a first coil which is electrically connected to an enzyme electrode and a reference electrode and transmits a signal generated via an enzyme reaction on the enzyme electrode; and a second coil which is placed proximate to the first coil and is connected to a detection device which receives the signal from the first coil, wherein said enzyme electrode comprises a direct electron transfer type oxidoreductase.

### FIELD OF THE INVENTION

The invention discloses the method of providing a novel scheme of monitoring target analyte concentration by measuring changes in a parallel resistance and capacitance of a near field communication antenna (NFC) employing direct electron transfer type enzymes.

### BACKGROUND OF THE INVENTION

The ultimate goal for those engaged in research to develop medical devices is to develop implantable biodevices, namely self-powered autonomously operated or battery free system, which do not require periodical replacement. Recently the idea of using new transduction modalities has become a forefront in enzyme biosensors. This includes using near field communication (NFC), which can passively measure changes in an antenna's capacitance wirelessly. Most self-powered enzyme fuel cell based sensors, and NFC based sensors rely on catalytic reactions which are limited based on needing sufficient surface area to enable enough electron transfer conversion between the anode and cathode. This also may cause these approaches to be limited by electroactive interference which may interact with the anode, as well as limit the time of use due to current flowing through the cathode. These limitations mostly revolve around the reliance of having current flowing through the anode to cathode via antenna pathway.

### SUMMARY OF THE INVENTION

In this invention, we disclose the development of a novel biocircuit realized the entirely passive target substrate monitoring, such as glucose and lactate, based on using capacitance measured over an antenna via NFC. The sensors are equipped with an electrode immobilized with redox-enzyme which is capable to transfer directly with electrode, and Ag/AgCl were connected in parallel to an antenna. As a result, the changes in parallel capacitance and resistance correlated well on the substrate of redox enzyme present in the solution. Using NFC as the transducer lends itself to being low power and can be easily miniaturized for both wearable and implantable form factors.

The sensor measured the physiological range of glucose and lactate with high resolution and showed good signal stability over several hours of use. Further, since our group's proposed system does not require catalytic turnover to maintain a signal, we have ability to measure using microelectrodes giving a signal the maintained fidelity over a wide range of electrode area.

### One Aspect of the Invention is to Provide a Biosensor Comprising:

a first coil which is electrically connected to an enzyme electrode and a reference electrode and transmits a signal generated via an enzyme reaction on the enzyme electrode; and
a second coil which is placed proximate to the first coil and is connected to a detection device which receives the signal from the first coil, wherein said enzyme electrode comprises a direct electron transfer type oxidoreductase.

In one embodiment, said direct electron transfer type oxidoreductase comprises an electron transfer subunit and/or electron transfer domain.

In one embodiment, said direct electron transfer type oxidoreductase comprises a cytochrome containing subunit.

In one embodiment, said direct electron transfer type oxidoreductase is selected from the group consisting of glucose dehydrogenase, lactate dehydrogenase, sorbitol dehydrogenase, D-fructose dehydrogenase, D-glucoside-3-dehydrogenase, and cellobiose dehydrogenase.

In one embodiment, said direct electron transfer type oxidoreductase is an oxidoreductase which is modified with an electron acceptor.

In one embodiment, said electron acceptor is selected from the group consisting of 1,4-naphthoquinone, vitamin K3, 9,10-phenanthrenequinone, 1,2-naphthoquinone, p-xyloquinone, methylbenzoquinone, 2,6-dimethylbenzoquinone, sodium 1,2-naphthoquinone-4-sulfonate, 1,4-anthraquinone, tetraethylbenzoquinone, and thymoquinone), phenylenediamine compounds (for example, N,N-dimethyl-1,4-phenylenediamine and N,N,N′,N′-tetramethyl-1,4-(phenazine ethosulfate), coenzyme Q0, AZURE A chloride, phenosafranin, 6-aminoquinoxaline, and tetrathiafulvalene.

In one embodiment, said biosensor is a glucose sensor or a lactate sensor.

In one embodiment, said detection device is a network analyzer which can detect a resistance change and/or a capacitance change.

Another aspect of the invention is to provide a method of measuring a substance comprising bringing a sample containing a substance to the biosensor as described above and measuring a signal generated by a reaction between the enzyme and the substance.

In one embodiment, said signal is a resistance change and/or a capacitance change.

In one embodiment, said measuring step is conducted at a frequency of 800 KHz to 30 MHz.

### BRIEF DESCRIPTION OF DRAWINGS

FIG. 1: Overall schematic of NFC based glucose sensor using direct electron transfer type enzyme

FIG. 2: Output of NanoVNA, showing Smith chart, and Polar Plot of antenna. For glucose detection analysis, the Smith plot was used. NanoVNA output displays the series and parallel resistance, and capacitance. Both of which correspond to changes in the antenna resistance and capacitance due to enzyme-substrate interactions.

FIG. 3: Overlay of two smith plot figures, showing the location of the two frequencies of interest (arrows at 15.03 MHz and 25.13 MHz) which showed glucose dependent response. The y axis is S11 (losses), which is related to the reflection coefficient. The X-axis is frequency in MHz.

FIG. 4: Calibration curves for Parallel resistance, and capacitance at two Hz of interest (15.03 and 25.13 MHz). a and b) parallel capacitance and resistance calibration curves at 15.03 MHz for glucose. c and d) parallel capacitance and resistance calibration curve at 25.13 MHz for glucose.

FIG. 5: a) and b) drift of signal at 3 Hz (15.03, 25.13 and 38.76 MHz) at 25 mM glucose over 3 hours. c) and d) show the parallel capacitance at 15.03 and 25.13 MHz comparing the blank with the adding of 25 mM glucose, as well as 3 hour drift, as well as the baseline signal of a blank electrode with BSA immobilized instead of glucose dehydrogenase.

### DETAILED DESCRIPTION

### a Biosensor According to One Embodiment of the Invention Comprises:

a first coil which is electrically connected to an enzyme electrode and a reference electrode and transmits a signal generated via an enzyme reaction on the enzyme electrode; and
a second coil which is placed proximate to the first coil and is connected to a detection device which receives the signal from the first coil, wherein said enzyme electrode comprises a direct electron transfer type oxidoreductase.

### <Enzyme Electrode>

The enzyme electrode comprises an oxidoreductase disposed on an electrode material. The electrode can be formed using a metallic material or a carbon material, wherein examples of the metallic material include gold (Au), platinum (Pt), silver (Ag), and palladium (Pd), and examples of the carbon material include carbons such as graphite, carbon nanotube, graphene, and mesoporous carbon. The electrode may be provided on an insulating substrate formed with an insulating material, wherein examples of the insulating material include resins (plastics) such as thermoplastic resins including polyetherimide (PEI), polyethylene terephthalate (PET), and polyethylene (PE), as well as polyimide resins and epoxy resins; glasses; ceramics; and papers.

### <Oxidoreductase>

The oxidoreductase may be selected depending on the type of the target substance. Examples of oxidoreductases include dehydrogenases such as alcohol dehydrogenase, glutamate dehydrogenase, cholesterol dehydrogenase, aldehyde dehydrogenase, glucose dehydrogenase (GDH), cellobiose dehydrogenase, fructose dehydrogenase, sorbitol dehydrogenase, lactate dehydrogenase, malate dehydrogenase, glycerol dehydrogenase, 17B hydroxysteroid dehydrogenase, estradiol 17B dehydrogenase, amino acid dehydrogenase, glyceraldehyde 3-phosphate dehydrogenase, 3-hydroxysteroid dehydrogenase; oxidases such as glucose oxidase (GOD), galactose oxidase, bilirubin oxidase, pyruvate oxidase, D- or L-amino acid oxidase, amine oxidase, cholesterol oxidase, choline oxidase, xanthine oxidase, sarcosine oxidase, L-lactate oxidase, ascorbate oxidase, fructosyl amino acid/peptide oxidases, glycerol-3phosphate oxidase; other oxidoreductases such as diaphorase, cytochrome oxidoreductase, catalase, peroxidase, and glutathione reductase.

The oxidoreductases may be oxidoreductases which uses coenzymes such as flavin adenine dinucleotide (FAD), flavin mononucleotide (FMN), pyrroloquinoline quinone (PQQ), or copper.

Depending on the type of the enzyme, the biosensor of the present invention can be used as a glucose sensor, cholesterol sensor, ethanol sensor, sorbitol sensor, fructose sensor, cellobiose sensor, lactate sensor, uric acid sensor, or the like.

Among these, an oxidoreductase capable of direct electron transfer with the electrode, that is, an oxidoreductase capable of direct transfer of electrons generated by the enzymatic reaction to the electrode without requiring an oxidation-reduction substance such as an electron acceptor (which oxidoreductase is also referred to as direct electron transfer-type oxidoreductase) is used. Examples of the oxidoreductase capable of direct electron transfer with the electrode include oxidoreductases physiologically including an oxidation-reduction molecule involved in electron transfer with the electrode. For example, an oxidoreductase containing an electron transfer subunit or an electron transfer domain as the oxidation-reduction molecule may be used. Examples of the electron transfer subunit include heme-containing subunits, and examples of the electron transfer domain include heme-containing domains. Examples of the heme-containing subunits and domains include subunits and domains containing home C or heme b, more specifically, subunits and domains containing a cytochrome such as cytochrome C or cytochrome b.

Examples of the enzyme containing a cytochrome-containing subunit as an electron transfer subunit include glucose dehydrogenase (GDH), sorbitol dehydrogenase (sorbitol DH), D-fructose dehydrogenase (fructose DH), D-glucoside-3-dehydrogenase, cellobiose dehydrogenase, lactate dehydrogenase, and urate oxidase.

Specific examples of the glucose dehydrogenase containing a cytochrome include cytochrome glucose dehydrogenase having an FAD-containing catalytic subunit (α-subunit) and a cytochrome subunit (β-subunit) (FADGDH), wherein the FADGDH preferably further has a regulatory subunit (γ-subunit) (FADGDH γαβ). Examples of the FADGDH include FAD-dependent glucose dehydrogenase derived from Burkholderia cepacia, and mutants thereof. The nucleotide sequence encoding the gamma subunit, alpha subunit, and beta subunit of FADGDH from B. cepacia is shown in SEQ ID NO: 1. The amino acid sequences of the gamma subunit, alpha subunit, and beta subunit of FADGDH from B. cepacia are shown in SEQ ID NOs: 2, 3 and 4, respectively. Examples of the mutants of FADGDH derived from Burkholderia cepacia include FADGDH mutants such as an α-subunit mutant in which the amino acid residues at positions 472 and 475 are substituted (WO 2005/103248), an α-subunit mutant in which the amino acid residues at positions 326, 365, and 472 are substituted (QYY: JP 2012-090563 A), and an α-subunit mutant in which the amino acid residues at positions 365, 326, 472, 475, 529, and the like are substituted (WO 2006/137283).

Direct electron transfer-type oxidoreductases also include an oxidoreductase which is modified with an electron acceptor and is capable of electron transfer with the electrode. The above-exemplified oxidoreductases can be modified with an electron acceptor. The electron acceptor herein may be a compound having no catalytic action which receives an electron from an oxidoreductase to undergo reduction, followed by reoxidization at the electrode. Examples of the electron acceptor include quinone compounds (for example, 1,4-naphthoquinone, vitamin K3, 9,10-phenanthrenequinone, 1,2-naphthoquinone, p-xyloquinone, methylbenzoquinone, 2,6-dimethylbenzoquinone, sodium 1,2-naphthoquinone-4-sulfonate, 1,4-anthraquinone, tetraethylbenzoquinone, and thymoquinone), phenylenediamine compounds (for example, N,N-dimethyl-1,4-phenylenediamine and N,N,N′,N′-tetramethyl-1,4-phenylenediamine), 1-methoxy-PMS (1-methoxy-5-methylphenazinium methylsulfate), PES (phenazine ethosulfate), coenzyme Q0, AZURE A chloride, phenosafranin, 6-aminoquinoxaline, and tetrathiafulvalene.

Examples of the method for the modification of the oxidoreductase with the electron acceptor include a method in which the electron acceptor is chemically bound to the enzyme (Biosensors and Bioelectronics 2020, 151, 111974). For example, the method may be a method in which a functional group such as succinimide is introduced to the electron acceptor, and the functional group is then reacted with an amino group of the enzyme to introduce the electron acceptor to the enzyme.

Examples of the method for arranging the direct electron transfer-type oxidoreductase on the electrode surface include, but are not limited to, a method in which the oxidoreductase is chemically immobilized on the electrode, a method in which the oxidoreductase is indirectly immobilized on the electrode using a conductive polymer, cross-linking agent, or the like (for example, WO 2014/002999 or JP 2016-121989 A), and a method in which the enzyme is immobilized on the electrode through a monolayer-forming molecule. Examples of the method in which the enzyme is immobilized on the electrode through a monolayer-forming molecule include a method in which the enzyme is immobilized on the electrode through a monolayer-forming molecule (SAM) disclosed in JP 2017-211383 A.

A monolayer forming molecule is a compound capable of binding to an electrode, and capable of binding the molecular recognition element, and is a compound capable of forming a monolayer when a plurality of the molecules are unidirectionally bound on the electrode surface. By using a monolayer forming molecule, the distance between the electrode and the enzyme molecule can be controlled.

The monolayer forming molecule preferably has a first functional group having affinity for the electrode, a spacer site, and a second functional group capable of reacting with a functional group of the molecular recognition element. More preferably it has a structure where the first functional group having affinity for the electrode is bound to the first end of the spacer site, and the second functional group capable of reacting with the functional group of the molecular recognition element is bound to the second end of the spacer site. Examples of the first functional group having affinity for the electrode include, when the electrode is metallic, a thiol group, and a dithiol group, and when the electrode is carbon, pyrene, and porphyrin.

Examples of the second functional group capable of reacting with a functional group of the molecular recognition element include a succinimide group when it is reacted with an amino group of the molecular recognition element (including the terminal amino group and the side chain amino group), and an oxazoline group when it is reacted with a carboxyl group of the molecular recognition element (including a terminal carboxyl group and a side chain carboxyl group).

Examples of a monolayer forming molecule having a thiol group, or a dithiol group include compounds having the following structures.

In this regard, L is a spacer, and X is a functional group capable of reacting with a functional group of the molecular recognition element. Examples thereof include a succinimide or its ester and a thiol. Examples of the type of spacer include alkylene having 1 to 20 (e.g. 3 to 7) carbon atoms, alkenylene having 1 to 20 (e.g. 3 to 7) carbon atoms, alkynylene having 1 to 20 (e.g. 3 to 7) carbon atoms, polyethylene glycol having a polymerization degree of 2 to 50, and an oligopeptide having 1 to 20 amino acid residues, or combinations of such spacers. In the alkylene, alkenylene, or alkynylene, one or more —CH₂— may be replaced by —O—.

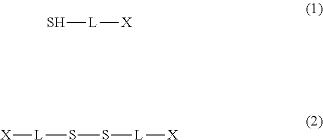

Examples of such a compound include the following DSH.

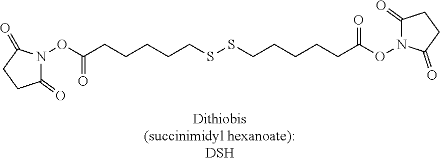 [SMILES: O=C(CCCCCSSCCCCCC(=O)ON1C(=O)CCC1=O)ON1C(=O)CCC1=O]

Examples of a monolayer forming molecule having, for example, pyrene or porphyrin include a compound having the following structure.

In this regard, Py stands for pyrene, Po for porphyrin, L for a spacer, and X for a functional group capable of reacting with the functional group of the enzyme molecule. Examples of the type of the spacer include alkylene having 1 to 20 carbon atoms, alkenylene having 1 to 20 carbon atoms, alkynylene having 1 to 20 carbon atoms, polyethylene glycol having a polymerization degree of 2 to 50, and an oligopeptide having 1 to 20 amino acid residues.

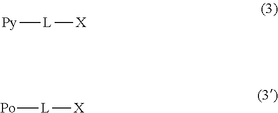

Examples of a monolayer forming molecule having, for example, pyrene include a compound having the following structure.

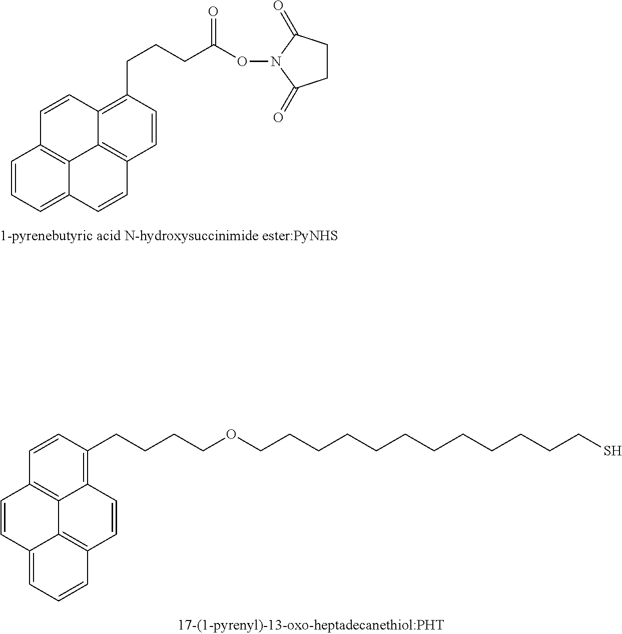 [SMILES: O=C(CCCc1ccc2ccc3cccc4ccc1c2c34)ON1C(=O)CCC1=O ; SCCCCCCCCCCCCOCCCCc1ccc2ccc3cccc4ccc1c2c34]

The enzyme electrode is prepared, for example, as follows.

First, a metal layer which functions as an electrode is formed on one side of an insulating substrate. For example, a metal layer having a desired thickness (for example, about 30 nm) is formed by depositing a metallic material, by physical vapor deposition (PVD, for example, sputtering) or chemical vapor deposition (CVD), on one side of an insulating substrate in the form of a film having a predetermined thickness (for example, about 100 μm). Instead of the metal layer, an electrode layer made of a carbon material may be formed.

To the surface of the thus obtained electrode layer, an enzyme is bound.

For example, in cases where a monolayer-forming molecule is used, the monolayer-forming molecule is first bound onto the electrode. Thereafter, by reacting a reactive functional group of the monolayer-forming molecule with an amino group or a carboxyl group of an oxidoreductase, the oxidoreductase can be immobilized on the electrode through the monolayer-forming molecule.

In cases where the enzyme is immobilized onto the electrode using a conductive polymer or a cross-linking agent, the enzyme and the reagent such as a conductive polymer or a cross-linking reagent may be added onto the electrode to prepare an enzyme electrode.

### <Reference/Counter Electrode>

The biosensor according to one embodiment of the invention also comprises a reference/counter electrode that forms a pair with the enzyme electrode. The reference/counter electrode is not limited as long as it can be generally used as a reference/counter electrode for a biosensor. Examples of the reference/counter electrode include a silver/silver chloride electrode and a calomel electrode.

### <Biosensor>

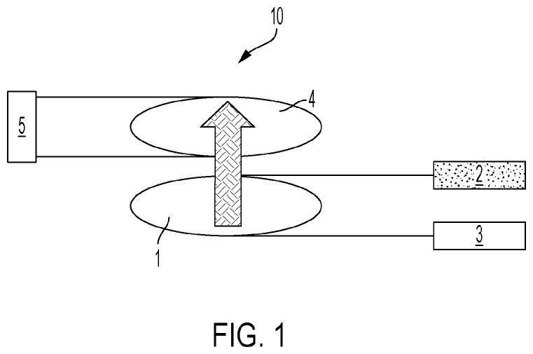

Hereinafter, one embodiment of the biosensor is explained with reference to FIG. 1.

However, the biosensor of the invention is not limited to this embodiment.

FIG. 1 shows the layout of the 2 coils, and how both the sensor, and NanoVNA interacted with the NFC system. As shown in FIG. 1, the biosensor (10) comprises a first coil (1) and a second coil (4).

The first coil (1) is connected to an enzyme electrode (2) and a reference electrode (3) and transmits a signal generated by the reaction of the enzyme (direct-electron transfer type oxidoreductase) on the enzyme electrode.

The second coil (4) is positioned close to the first coil and receives the signal from the first coil, which is detected by a detector (5) connected to the second coil.

The detector may be a network analyzer which can measure capacitance and/o resistance of an electrical network. An example of the network analyzer includes VNA.

### NanoVNA User Guide | NanoVNA User Guide

cho45.github.io/Nano VNA-manual/

When a measurement sample containing a measuring target substance, which is a substrate for the enzyme, is applied on the enzyme electrode, the measuring target substance is reacted with the enzyme. As a result of the reaction, an electron is generated and transferred to the electrode, thereby a signal is transmitted from the first coil.

The second coil receives this signal and the detector connected to the second soil detects a capacitance change or a resistance change both of which is dependent on the signal.

The capacitance change and resistance change depend on the reaction, that is, depend on the measuring target substance in the sample, and thus the amount of the measuring target substance can be calculated based on the amount (value) of the capacitance change or the resistance change.

It is preferable that a calibration curve which shows the relationship between the amount of the measuring target substance and the value of the capacitance change or the resistance change is prepared beforehand.

For measuring the signal such as capacitance change and/or resistance change, it is preferable that the frequency of the detector is adjusted in the range of 800 KHz to 30 MHz.

The first coil and the second coil may be a conventional coil having electric conductivity such as copper.

Measuring target substance is not limited as long as it is a substrate of the enzyme (oxidoreductase) and examples thereof include glucose, cholesterol, ethanol, sorbitol, fructose, cellobiose, lactate, uric acid, and the like.

Samples used for measurement are not limited as long as they contain the measuring target substance exemplified above and examples thereof include a blood sample, an urine sample, a sweat sample, an interstitial fluid sample or a saliva sample, a culture medium, an environmental water and the like.

The biosensor is available both for self-measurement and for continuous measurement.

### EXAMPLES

Recombinant Burkholderia cepacia glucose dehydrogenase (BcGDH) comprising γαβ subunits were prepared using expression vector pTre99A containing the structural gene for the His-tagged BcGDH according to the method in previous study for heme c maturation^{1,2}. These vectors were co-transformed into Escherichia coli strain BL21 (DE3) and cultivated according to our previous reports^{2,3}. Co-transformed E. coli were cultured in 500 mL conical flasks containing 100 mL of ZYP-5052 medium (Protein Express. Purif. 41, 207-234 (2005)) in a rotary shaker at 30° C. for 30 h. Cells were then harvested by centrifugation, and disrupted by sonication in 10 mM HEPES buffer pH 7.8 containing 10 mM Imidazole, 500 mM NaCl, and 1% Sodium cholate. The lysate was centrifuged at 16,000×g for 20 min and the supernatant was then applied to supercentrifuge at 29,000×g for 60 min. The supernatant was applied to HisTrap HP column (Cytiva) equilibrated with 10 mM HEPES pH 7.8 containing 10 mM Imidazole, 500 mM NaCl, and 1% Sodium cholate. Protein was eluted with linear Imidazole gradient in same buffer. The active fraction were pooled and dialyzed overnight at 4° C. against PBS containing 1% sodium cholate and applied to Superdex-200 increase 10/300GL (Cytiva) equilibrated with PBS containing 1% sodium cholate. Pooled fractions were dialyzed at 4° C. against 10 mM potassium phosphate buffer containing, pH 7.0. Purity of purified protein was confirmed by SDS-PAGE.

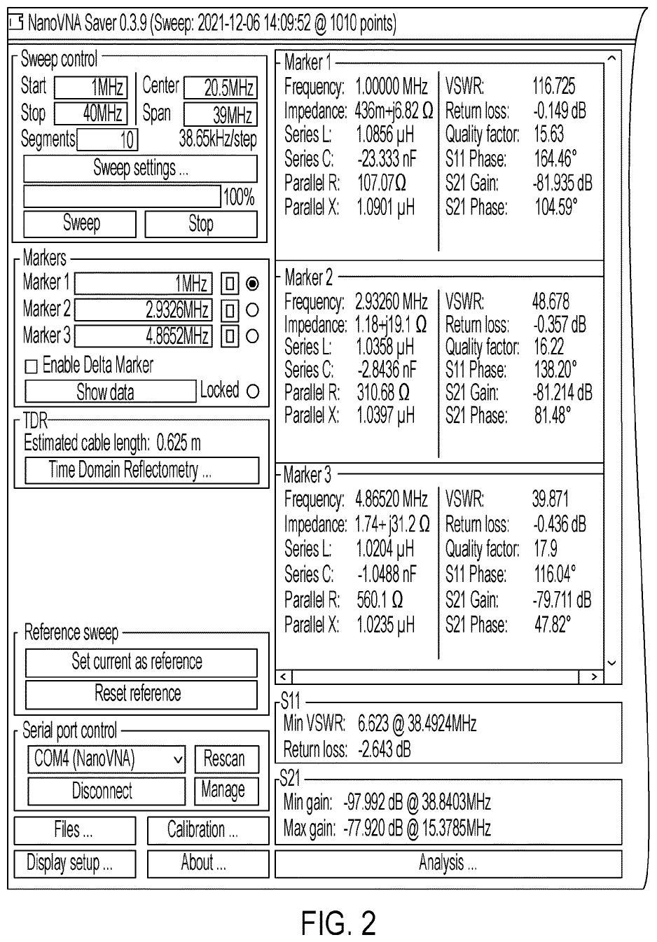

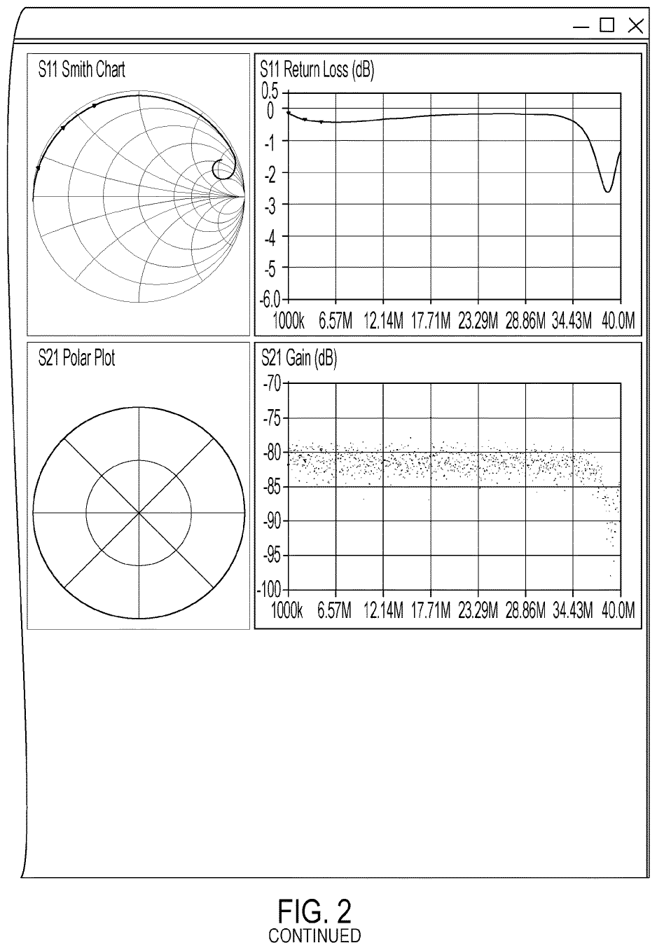

A Nano Vector Network Analyzer NanoVNA was used for investigating the change in parallel resistance and capacitance. For both Glucose and Lactate 2 mm diameter gold disc electrodes were used, first polished by 0.3 μm, and 0.05 μm aluminum oxide. All electrodes were then chemically cleaned using piranha (3:1 ratio of sulfuric acid and hydrogen peroxide), followed by electrochemical polishing by applying cyclic voltammetry over the electrode sweeping between-1.2-0.0 V immersed in 50 mM potassium hydroxide. DSH was then immobilized to each electrode using by soaking in 100 μM solution, at room temperature, overnight. BcGDH was then incubated for enzyme immobilization onto each electrode. Testing was performed by creating 2 coils (3 inches each in diameter) by wrapping wire around a 3-inch diameter cylinder 4 times over. The coils were then overlaid on top of one another separated by only 2 wraps of tape approximately 1-2 mm apart. One coil was then connected to the input/output NanoVNA, while the second coil was connected to the working and reference/counter electrode (Ag/AgCl was used as the counter and refence electrode for all experiments). The 2 electrodes were then submerged in 10 mM potassium phosphate buffer solution (PPB) to ensure a closed system. Glucose was serially added to the solution, and the respective parallel capacitance or resistance was measured 9 times, removing the highest, and lowest values, giving an average of n=7 per reading. Frequencies were swept from 0.8-40 MHz at 38.65 KHz/step, measuring the total impedance of the antenna by means of parallel capacitance, and resistance. Results for each sweep of the NanoVNA were captured as shown in FIG. 2.

A Nano Vector Network Analyzer (NanoVNA) was used for investigating the change in parallel resistance and capacitance. For both Glucose and Lactate 2 mm diameter gold disc electrodes were used, first polished by 0.3 μm, and 0.05 μm aluminum oxide. All electrodes were then chemically cleaned using piranha (3:1 ratio of sulfuric acid and hydrogen peroxide), followed by electrochemical polishing by applying cyclic voltammetry over the electrode sweeping between ~1.2-0.0 V immersed in 50 mM potassium hydroxide. DSH was then immobilized to each electrode using by soaking in 100 M solution, at room temperature, overnight. DET-type GDH (FAD-GDH from B. cepacia) was then incubated for enzyme immobilization onto each electrode. Testing was performed by creating 2 coils (3 inches each in diameter) by wrapping wire around a 3-inch diameter cylinder 4 times over. The coils were then overlaid on top of one another separated by only 2 wraps of tape. One coil was then connected to the input/output NanoVNA, while the second coil was connected to the working and reference/counter electrode (Ag/AgCl was used as the counter and refence electrode for all experiments). The 2 electrodes were then submerged in 10 mM potassium phosphate buffer solution (PPB) to ensure a closed system. Glucose was serially added to the solution, and the respective parallel capacitance or resistance was measured 9 times, removing the highest, and lowest values, giving an average of n=7 per reading.

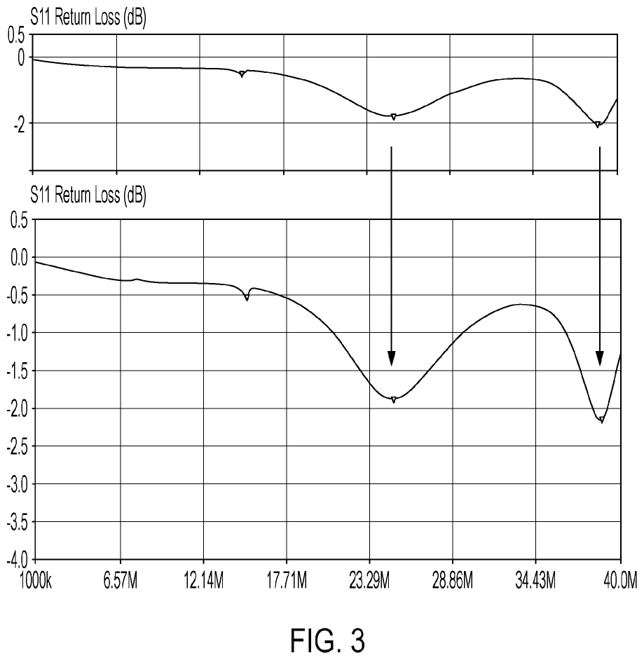

FIG. 3 shows a smith chart, allowing the analyzes of the Reflection Coefficient:

$$Γ = \frac{{V}_{Reflected}}{{V}_{Incident}} = \frac{Z - {Z}_{0}}{Z + {Z}_{0}}$$

Where Z is the impedance of the system being measured. The reflection coefficient is directly related to the S11 losses for the antenna, which is a function of the change in total impedance between the two coils (better matching impedance will give better performance of the antenna). With the addition of glucose, there is a massive chance to the total impedance (capacitive, and resistive) which is in part caused by the reduction of FADGDH on the electrode surface. Based on the change in peaks in FIG. 3 we chose to move forward analyzing 15.03 and 25.13 MHz in future experiments.

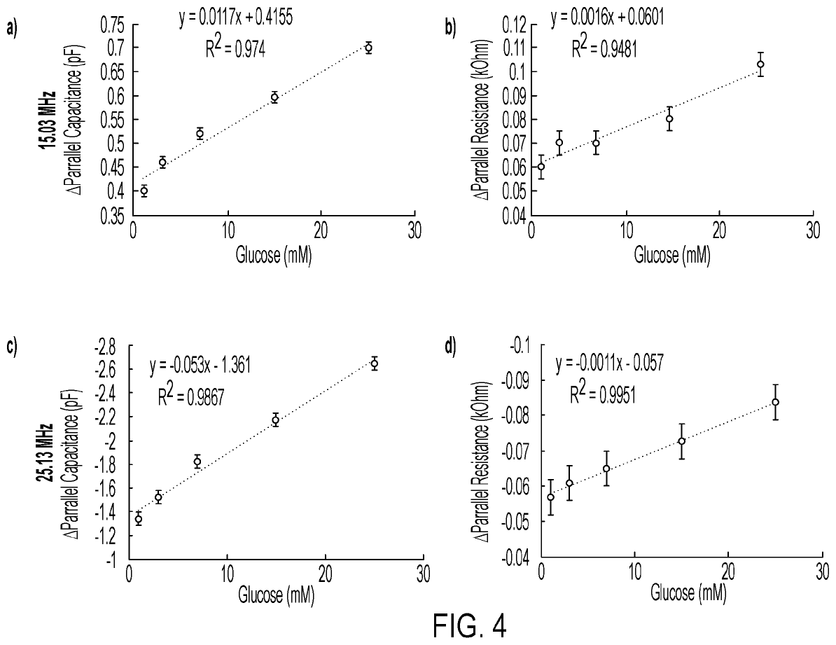

FIG. 4, panels a-d shows the change in Δparallel capacitance, and resistance across 2 different frequencies. Δparallel capacitance, and resistance were calculated by taking the change in either capacitance, and resistance against the blank (0 mM glucose). FIG. 4, panel a shows the Δparallel capacitance calibration curve for glucose measured at 15.03 MHz, giving a slope of 0.0117 pF/mM glucose, with a correlation of 0.974. FIG. 4, panel b Δparallel resistance calibration curve for glucose measured at 15.03 MHz, giving a slope of 0.0016 KOhm/mM, and a correlation of 0.948. FIG. 4, panel c shows the Δparallel capacitance calibration curve for glucose measured at 15.03 MHz, giving a slope of −0.053 pF/mM glucose, with a correlation of 0.987. FIG. 4, panel d Δparallel resistance calibration curve for glucose measured at 15.03 MHz, giving a slope of −0.0011 KOhm/mM, and a correlation of 0.995. These frequencies were selected by using the NanoVNA and sweeping over a wide range of MHz (0.8-40) and adding glucose form 0 mM to 25 mM. As glucose was added, two key peaks appeared at both 15.03, and 25.13 MHz. This may be attributed to the specific coil, and the ideal frequency for measurement will change based on the capacitive, resistive, and inductive qualities of the implemented coil.

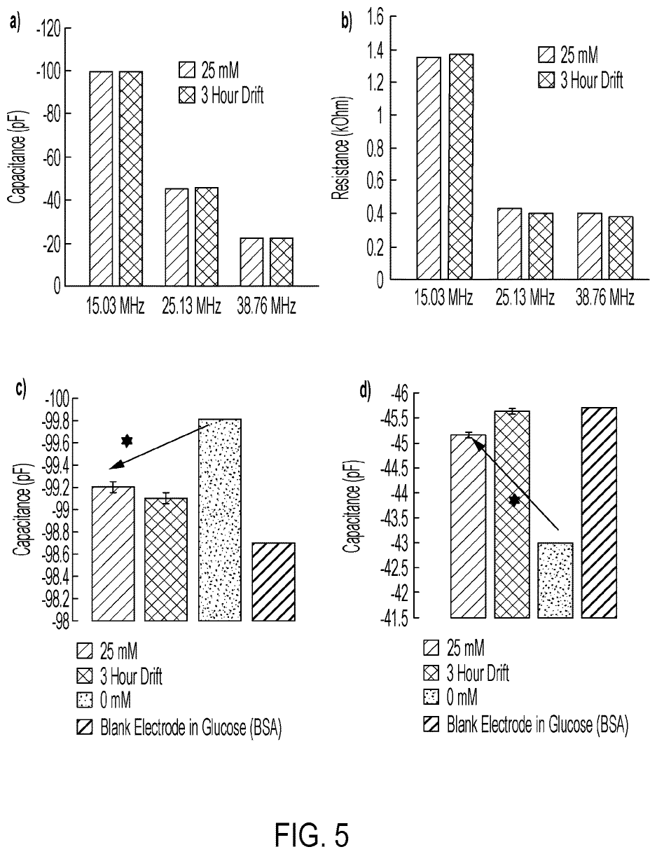

FIG. 5, panels a-d shows the stability of the sensor and compares the impact of drift over both the absolute capacitance, and resistance. FIG. 5, panels c and d show how the change in capacitance is due to the interaction of glucose, and FADGDH, not drift. FIG. 5, panel c is measured at 15.03 MHz, while FIG. 5, panel d is measured at 25.13 MHz. Upon the addition of glucose there is a statistically significant change in capacitance with a P value <0.005 at either frequency. Also shown in the figure is a blank electrode immobilized with BSA instead of FADGDH. As seen in FIG. 5, panels c and d there is no change in the BSA immobilized sensor, only the FADGDH. Further showing that the change in total impedance is due to the specific interaction of glucose and then enzyme.

The contents of all publications including patents and patent applications as well as non-patent documents cited in the present description are hereby incorporated by reference to the same extent as if all contents were clearly described.

### REFERENCES

(1) Tsuya, T.; Ferri, S.; Fujikawa, M.; Yamaoka, H.; Sode, K. Cloning and Functional Expression of Glucose Dehydrogenase Complex of Burkholderia Cepacia in Escherichia Coli. Journal of Biotechnology 2006, 123 (2), 127-136. doi.org/10.1016/j.jbiotec.2005.10.017.
(2) Yamashita, Y.; Suzuki, N.; Hirose, N.; Kojima, K.; Tsugawa, W.; Sode, K. Mutagenesis Study of the Cytochrome c Subunit Responsible for the Direct Electron Transfer-Type Catalytic Activity of FAD-Dependent Glucose Dehydrogenase. IJMS 2018, 19 (4), 931. doi.org/10.3390/ijms19040931.
(3) Yoshida, H.; Kojima, K.; Shiota, M.; Yoshimatsu, K.; Yamazaki, T.; Ferri, S.; Tsugawa, W.; Kamitori, S.; Sode, K. X-Ray Structure of the Direct Electron Transfer-Type FAD Glucose Dehydrogenase Catalytic Subunit Complexed with a Hitchhiker Protein. Acta Crystallogr D Struct Biol 2019, 75 (9), 841-851. doi.org/10.1107/S2059798319010878.

What is claimed is:

1. A biosensor comprising:
a first coil which is electrically connected to an enzyme electrode and a reference electrode and transmits a signal generated via an enzyme reaction on the enzyme electrode; and
a second coil which is placed proximate to the first coil and is connected to a detection device which receives the signal from the first coil, wherein said enzyme electrode comprises a direct electron transfer type oxidoreductase.

2. The biosensor according to claim 1, wherein said direct electron transfer type oxidoreductase comprises an electron transfer subunit and/or electron transfer domain.

3. The biosensor according to claim 1, wherein said direct electron transfer type oxidoreductase comprises a cytochrome containing subunit.

4. The biosensor according to claim 1, wherein said direct electron transfer type oxidoreductase is selected from the group consisting of glucose dehydrogenase, lactate dehydrogenase, sorbitol dehydrogenase, D-fructose dehydrogenase, D-glucoside-3-dehydrogenase, and cellobiose dehydrogenase.

5. The biosensor according to claim 1, wherein said direct electron transfer type oxidoreductase is an oxidoreductase which is modified with an electron acceptor.

6. The biosensor according to claim 1, wherein said electron acceptor is selected from the group consisting of 1,4-naphthoquinone, vitamin K3, 9,10-phenanthrenequinone, 1,2-naphthoquinone, p-xyloquinone, methylbenzoquinone, 2,6-dimethylbenzoquinone, sodium 1,2-naphthoquinone-4-sulfonate, 1,4-anthraquinone, tetramethylbenzoquinone, and thymoquinone), phenylenediamine compounds (for example, N,N-dimethyl-1,4-phenylenediamine and N,N,N′,N′-tetramethyl-1,4-(phenazine ethosulfate), coenzyme Q0, AZURE A chloride, phenosafranin, 6-aminoquinoxaline, and tetrathiafulvalene.

7. The biosensor according to claim 1, wherein said biosensor is a glucose sensor or a lactate sensor.

8. The biosensor according to claim 1, wherein said detection device is a network analyzer which can detect a resistance change and/or a capacitance change.

9. A method of measuring a substance comprising bringing a sample containing a substance to the biosensor according to claim 1 and measuring a signal generated by a reaction between the enzyme and the substance.

10. The method according to claim 9, wherein said signal is a resistance change and/or a capacitance change.

11. The method according to claim 9, wherein said measuring step is conducted at a frequency of 800 KHz to 30 MHz.


SEQUENCE LISTING (4 sequences)

SEQ ID NO: 1 - DNA, 3706 bp, Burkholderia cepacia
  source 1..3706: mol_type = genomic DNA
  CDS 258..761: protein_id = 2; translation = MHNDNTPHSRRHGDAAASGITRRQWLQGALALTAAGLTGSLTLR ALADNPGTAPLDTFMTLSESLTGKKGLSRVIGERLLQALQKGSFKTADSLPQLAGALA SGSLTPEQESLALTILEAWYLGIVDNVVITYEEALMFGVVSDTLVIRSYCPNKPGFWA DKPIERQA
  CDS 764..2380: protein_id = 3; translation = MADTDTQKADVVVVGSGVAGAIVAHQLAMAGKAVILLEAGPRMP RWEIVERFRNQPDKMDFMAPYPSSPWAPHPEYGPPNDYLILKGEHKFNSQYIRAVGGT TWHWAASAWRFIPNDFKMKSVYGVGRDWPIQYDDLEPYYQRAEEELGVWGPGPEEDLY SPRKQPYPMPPLPLSFNEQTIKTALNNYDPKFHVVTEPVARNSRPYDGRPTCCGNNNC MPICPIGAMYNGIVHVEKAERAGAKLIENAVVYKLETGPDKRIVAALYKDKTGAEHRV EGKYFVLAANGIETPKILLMSANRDFPNGVANSSDMVGRNLMDHPGTGVSFYASEKLW PGRGPQEMTSLIGFRDGPFRATEAAKKIHLSNLSRIDQETQKIFKAGKLMKPDELDAQ IRDRSARYVQFDCFHEILPQPENRIVPSKTATDAIGIPRPEITYAIDDYVKRGAAHTR EVYATAAKVLGGTDVVFNDEFAPNNHITGSTIMGADARDSVVDKDCRTFDHPNLFISS SATMPTVGTVNVTLTIAALALRMSDTLKKEV
  CDS 2386..3660: protein_id = 4; translation = VRKSTLTFLIAGCLALPGFARAADAADPALVKRGEYLATAGDCM ACHTVKGGKPYAGGLGMPVPMLGKIYTSNITPDPDTGIGKWTFEDFERAVRHGVSKNG DNLYPAMPYVSYAKITDDDVRALYAYFMHGVEPVKQAPPKNEIPALLSMRWPLKIWNW LFLKDGPYQPKPSQSAEWNRGAYLVQGLAHCSTCHTPRGIAMQEKSLDETGGSFLAGS VLAGWDGYNITSDPNAGIGSWTQQQLVQYLRTGSVPGVAQAAGPMAEAVEHSFSKMTE ADIGAIATYVRTVPAVADSNAKQPRSSWGKPAEDGLKLRGVALASSGIDPARLYLGNC ATCHQMQGKGTPDGYYPSLFHNSTVGASNPSNLVQVILNGVQRKIGSEDIGMPAFRYD LNDAQIAALTNYVTAQFGNPAAKVTEQDVAKLR
aagctttctgtttgattgcacgcgattctaaccgagcgtctgtgaggcggaacgcgacatgcttcgtgtcgcacacgtgtcgcgccgacgacacaaaaatgcagcgaaatggctgatcgttacgaatggctgacacattgaatggactataaaaccattgtccgttccggaatgtgcgcgtacatttcaggtccgcgccgatttttgagaaatatcaagcgtggttttcccgaatccggtgttcgagagaaggaaacatgcacaacgacaacactccccactcgcgtcgccacggcgacgcagccgcatcaggcatcacgcggcgtcaatggttgcaaggcgcgctggcgctgaccgcagcgggcctcacgggttcgctgacattgcgggcgcttgcagacaaccccggcactgcgccgctcgatacgttcatgacgctttccgaatcgctgaccggcaagaaagggctcagccgcgtgatcggcgagcgcctgctgcaggcgctgcagaagggctcgttcaagacggccgacagcctgccgcagctcgccggcgcgctcgcgtccggttcgctgacgcctgaacaggaatcgctcgcactgacgatcctcgaggcctggtatctcggcatcgtcgacaacgtcgtgattacgtacgaggaagcattaatgttcggcgtcgtgtccgatacgctcgtgatccgttcgtattgccccaacaaacccggcttctgggccgacaaaccgatcgagaggcaagcctgatggccgataccgatacgcaaaaggccgacgtcgtcgtcgttggatcgggtgtcgcgggcgcgatcgtcgcgcatcagctcgcgatggcgggcaaggcggtgatcctgctcgaagcgggcccgcgcatgccgcgctgggaaatcgtcgagcgcttccgcaatcagcccgacaagatggacttcatggcgccgtacccgtcgagcccctgggcgccgcatcccgagtacggcccgccgaacgactacctgatcctgaagggcgagcacaagttcaactcgcagtacatccgcgcggtgggcggcacgacgtggcactgggccgcgtcggcgtggcgcttcattccgaacgacttcaagatgaagagcgtgtacggcgtcggccgcgactggccgatccagtacgacgatctcgagccgtactatcagcgcgcggaggaagagctcggcgtgtggggcccgggccccgaggaagatctgtactcgccgcgcaagcagccgtatccgatgccgccgctgccgttgtcgttcaacgagcagaccatcaagacggcgctgaacaactacgatccgaagttccatgtcgtgaccgagccggtcgcgcgcaacagccgcccgtacgacggccgcccgacttgttgcggcaacaacaactgcatgccgatctgcccgatcggcgcgatgtacaacggcatcgtgcacgtcgagaaggccgaacgcgccggcgcgaagctgatcgagaacgcggtcgtctacaagctcgagacgggcccggacaagcgcatcgtcgcggcgctctacaaggacaagacgggcgccgagcatcgcgtcgaaggcaagtatttcgtgctcgccgcgaacggcatcgagacgccgaagatcctgctgatgtccgcgaaccgcgatttcccgaacggtgtcgcgaacagctcggacatggtcggccgcaacctgatggaccatccgggcaccggcgtgtcgttctatgcgagcgagaagctgtggccgggccgcggcccgcaggagatgacgtcgctgatcggtttccgcgacggtccgttccgcgcgaccgaagcggcgaagaagatccacctgtcgaacctgtcgcgcatcgaccaggagacgcagaagatcttcaaggccggcaagctgatgaagcccgacgagctcgacgcgcagatccgcgaccgttccgcacgctacgtgcagttcgactgcttccacgaaatcctgccgcaacccgagaaccgcatcgtgccgagcaagacggcgaccgatgcgatcggcattccgcgccccgagatcacgtatgcgatcgacgactacgtgaagcgcggcgccgcgcatacgcgcgaggtctacgcgaccgccgcgaaggtgctcggcggcacggacgtcgtgttcaacgacgaattcgcgccgaacaatcacatcacgggctcgacgatcatgggcgccgatgcgcgcgactccgtcgtcgacaaggactgccgcacgttcgaccatccgaacctgttcatttcgagcagcgcgacgatgccgaccgtcggtaccgtaaacgtgacgctgacgatcgccgcgctcgcgctgcggatgtcggacacgctgaagaaggaagtctgaccgtgcggaaatctactctcactttcctcatcgccggctgcctcgcgttgccgggcttcgcgcgcgcggccgatgcggccgatccggcgctggtcaagcgcggcgaatacctcgcgaccgccggcgactgcatggcctgccacaccgtgaagggcggcaagccgtacgcgggcggccttggcatgccggtaccgatgctcggcaagatctacacgagcaacatcacgcccgatcccgatacgggcatcggcaaatggacgttcgaggacttcgagcgcgcggtgcggcacggcgtgtcgaagaacggcgacaacctgtatccggcgatgccgtacgtgtcgtacgcgaagatcacggacgacgacgtacgcgcgctgtacgcctacttcatgcacggcgtcgagccggtcaagcaggcgccgccgaagaacgagattcccgcgctgctcagcatgcgctggccgctgaagatctggaactggctgttcctgaaggacggcccgtaccagccgaagccgtcgcagagcgccgaatggaatcgcggcgcgtatctggtgcagggtctcgcgcactgcagcacgtgccacacgccgcgcggcatcgcgatgcaggagaagtcgctcgacgaaaccggcggcagcttcctcgcggggtcggtgctcgccggctgggacggctacaacatcacgtcggacccgaatgcggggatcggcagctggacgcagcagcagctcgtgcagtatttgcgcaccggcagcgtgccgggcgtcgcgcaggcggccgggccgatggccgaggcggtcgagcacagcttctcgaagatgaccgaagcggacatcggtgcgatcgccacgtacgtccgcacggtgccggccgttgccgacagcaacgcgaagcagccgcggtcgtcgtggggcaagccggccgaggacgggctgaagctgcgcggtgtcgcgctcgcgtcgtcgggcatcgatccggcgcggctgtatctcggcaactgcgcgacgtgccaccagatgcagggcaagggcacgccggacggctattacccgtcgctgttccacaactccaccgtcggcgcgtcgaatccgtcgaacctcgtgcaggtgatcctgaacggcgtgcagcgcaagatcggcagcgaggatatcgggatgcccgctttccgctacgatctgaacgacgcgcagatcgccgcgctgacgaactacgtgaccgcgcagttcggcaatccggcggcgaaggtgacggagcaggacgtcgcgaagctgcgctgacatagtcgggcgcgccgacacggcgcaaccgataggacaggag

SEQ ID NO: 2 - protein, 168 aa, Burkholderia cepacia
MHNDNTPHSRRHGDAAASGITRRQWLQGALALTAAGLTGSLTLRALADNPGTAPLDTFMTLSESLTGKKGLSRVIGERLLQALQKGSFKTADSLPQLAGALASGSLTPEQESLALTILEAWYLGIVDNVVITYEEALMFGVVSDTLVIRSYCPNKPGFWADKPIERQA

SEQ ID NO: 3 - protein, 539 aa, Burkholderia cepacia
MADTDTQKADVVVVGSGVAGAIVAHQLAMAGKAVILLEAGPRMPRWEIVERFRNQPDKMDFMAPYPSSPWAPHPEYGPPNDYLILKGEHKFNSQYIRAVGGTTWHWAASAWRFIPNDFKMKSVYGVGRDWPIQYDDLEPYYQRAEEELGVWGPGPEEDLYSPRKQPYPMPPLPLSFNEQTIKTALNNYDPKFHVVTEPVARNSRPYDGRPTCCGNNNCMPICPIGAMYNGIVHVEKAERAGAKLIENAVVYKLETGPDKRIVAALYKDKTGAEHRVEGKYFVLAANGIETPKILLMSANRDFPNGVANSSDMVGRNLMDHPGTGVSFYASEKLWPGRGPQEMTSLIGFRDGPFRATEAAKKIHLSNLSRIDQETQKIFKAGKLMKPDELDAQIRDRSARYVQFDCFHEILPQPENRIVPSKTATDAIGIPRPEITYAIDDYVKRGAAHTREVYATAAKVLGGTDVVFNDEFAPNNHITGSTIMGADARDSVVDKDCRTFDHPNLFISSSATMPTVGTVNVTLTIAALALRMSDTLKKEV

SEQ ID NO: 4 - protein, 425 aa, Burkholderia cepacia
VRKSTLTFLIAGCLALPGFARAADAADPALVKRGEYLATAGDCMACHTVKGGKPYAGGLGMPVPMLGKIYTSNITPDPDTGIGKWTFEDFERAVRHGVSKNGDNLYPAMPYVSYAKITDDDVRALYAYFMHGVEPVKQAPPKNEIPALLSMRWPLKIWNWLFLKDGPYQPKPSQSAEWNRGAYLVQGLAHCSTCHTPRGIAMQEKSLDETGGSFLAGSVLAGWDGYNITSDPNAGIGSWTQQQLVQYLRTGSVPGVAQAAGPMAEAVEHSFSKMTEADIGAIATYVRTVPAVADSNAKQPRSSWGKPAEDGLKLRGVALASSGIDPARLYLGNCATCHQMQGKGTPDGYYPSLFHNSTVGASNPSNLVQVILNGVQRKIGSEDIGMPAFRYDLNDAQIAALTNYVTAQFGNPAAKVTEQDVAKLR